(19) United States
(12) Patent Application Publication
(10) Pub. No.: US 2015/0133669 A1
(43) Pub. Date: May 14, 2015

(54) NEW PROCESS FOR THE PREPARATION OF 2-CYCLOPENTYL-6-METHOXY-ISONICOTINIC ACID

(71) Applicant: ACTELION PHARMACEUTICALS LTD, Allschwil (CH)

(72) Inventor: Gunther Schmidt, Allschwil (CH)

(73) Assignee: ACTELION PHARMACEUTICALS LTD., ALLSCHWIL (CH)

(21) Appl. No.: 14/402,159

(22) PCT Filed: May 21, 2013
(86) PCT No.: PCT/IB2013/054170

§ 371 Date: Nov. 19, 2014

(30) Foreign Application Priority Data
May 22, 2012 (EP) 12168823.8

(51) Int. Cl. C07C 45/67; C07D 413/04; C07D 213/803; C07D 213/79; C07C 67/343; C07D 213/85

(52) U.S. Cl. CPC C07C 45/676; C07C 67/343; C07D 213/85; C07D 213/803; C07D 213/79; C07D 413/04; C07C 2101/08
USPC 546/250; 568/346; 546/288; 546/298; 546/326; 546/269.4

(57) ABSTRACT

The present invention relates to new processes for the preparation of 2-cyclopentyl-6-methoxy-isonicotinic acid, which is a useful intermediate for the synthesis of pyridine-4-yl derivatives as immunomodulating agent. Moreover, the present invention also relates to new intermediates used in those processes.

### FIELD OF THE INVENTION

The present invention relates to new processes for the preparation of 2-cyclopentyl-6-methoxy-isonicotinic acid, which is a useful intermediate for the synthesis of pyridine-4-yl derivatives of formula (PD) disclosed in WO2011007324 as immunomodulating agent. Moreover, the present invention also relates to new intermediates used in those processes.

### BACKGROUND OF THE INVENTION

Pyridine-4-yl derivatives of formula (PD),

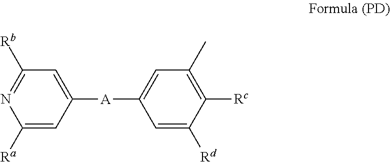

(wherein
A represents

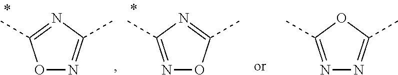

(the asterisks indicate the bond that is linked to the pyridine group of Formula (PD));
R^{a} represents 3-pentyl, 3-methyl-but-1-yl, cyclopentyl, or cyclohexyl;
R^{b} represents methoxy;
R^{c} represents 2,3-dihydroxypropoxy, —OCH₂—CH(OH)—CH₂—NHCO—CH₂OH, —OCH₂—CH(OH)—CH₂N(CH₃)—CO—CH(OH, —NHSO₂CH₃, or —NHSO₂CH₂CH₃; and
R^{d} represents ethyl or chloro.)
disclosed in WO2011007324, have immunomodulating activity through their S1P1/EDG1 receptor agonistic activity. Therefore, those pyridine-4-yl derivatives are useful for prevention and/or treatment of diseases or disorders associated with an activated immune system, including rejection of transplanted organs such as kidney, liver, heart, lung, pancreas, cornea, and skin; graft-versus-host diseases brought about by stem cell transplantation; autoimmune syndromes including rheumatoid arthritis, multiple sclerosis, inflammatory bowel diseases such as Crohn's disease and ulcerative colitis, psoriasis, psoriatic arthritis, thyroiditis such as Hashimoto's thyroiditis, uveo-retinitis; atopic diseases such as rhinitis, conjunctivitis, dermatitis; asthma; type I diabetes; post-infectious autoimmune diseases including rheumatic fever and post-infectious glomerulonephritis; solid cancers and tumor metastasis. 2-Cyclopentyl-6-methoxy-isonicotinic acid, which is also disclosed in WO2011007324, is a useful intermediate for the synthesis of the pyridine-4-yl derivatives of formula (PD), wherein R^{a} is a cyclopentyl group.

In the process described in WO2011007324, 2-cyclopentyl-6-methoxy-isonicotinic acid was prepared according to the following reaction scheme 1:

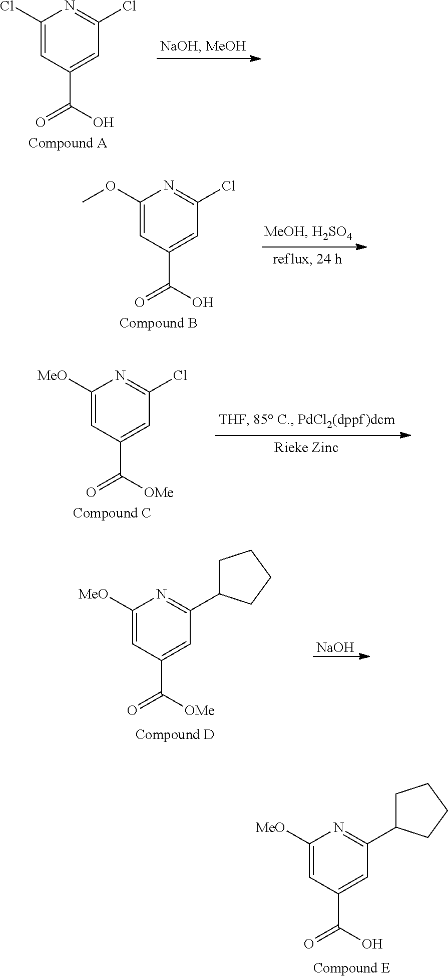 [SMILES: O=C(O)c1cc(Cl)nc(Cl)c1 ; COc1cc(C(=O)O)cc(Cl)n1 ; COC(=O)c1cc(Cl)nc(OC)c1 ; COC(=O)c1cc(OC)nc(C2CCCC2)c1 ; "NaOH" ; COc1cc(C(=O)O)cc(C2CCCC2)n1]

Rieke Zinc: cyclopentylzinc bromide;
PdCl₂(dppf)dcm: 1,1′-Bis(diphenylphosphino)ferrocene-palladium(II)dichloride dichloromethane complex

However, the abovementioned process has drawbacks for larger scale, i.e. industrial scale synthesis of 2-cyclopentyl-6-methoxy-isonicotinic acid, for the following reasons:

a) The commercially available starting material, 2,6-dichloro-isonicotinic acid (Compound A) is expensive.
b) The conversion of Compound C to Compound D is cost-intensive. The reaction has to be performed under protective atmosphere with expensive palladium catalysts and highly reactive and expensive Rieke zinc complex. Such synthesis steps are expensive to scale up and it was therefore highly desired to find alternative synthesis methods.

Even though Goldsworthy, J. Chem. Soc. 1934, 377-378 discloses the preparation of 1-cyclopentylethanone, which is a key building block in the new process of the present invention, by using ethyl 1-acetoacetate as a starting material, this synthesis was far from being suitable in an industrial process. The reported yield was low (see also under “Referential Examples” below).

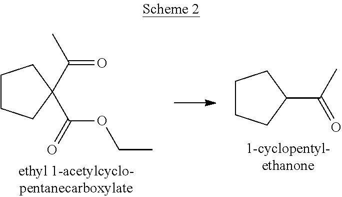

Besides the early work by Goldsworthy there are several recent examples for the preparation of 1-cyclopentylethanone described in the literature. Such examples include:
1) Addition of methyl lithium to a N-cyclopentanecarbonyl-N,O-dimethylhydroxylamine at −78° C. in a yield of 77%. US2006/199853 A1, 2006 and US2006/223884 A1, 2006.
2) Addition of methyl lithium to a cyclopentyl carboxylic acid in diethylether at −78° C. in a yield of 81%. J. Am. Chem. Soc., 1983,105, 4008-4017.
3) Addition of methylmagnesiumbromide to cyclopentanecarbonitrile. Bull. Soc. Chim. Fr., 1967, 3722-3729.
4) Oxidation of 1-cyclopentylethanol with chromtrioxide. U.S. Pat. No. 5,001,140 A1, 1991. WO2009/71707 A1, 2009.
5) Addition of cyclopentylmagnesium bromide to acetic anhydride at −78° C. with a yield of 54%. WO2004/74270 A2, 2004.
6) Synthesis of 1-cyclopentylethanone in 5 steps from cyclopentanone. Zhang, Pang; Li, Lian-chu, Synth. Commun., 1986, 16, 957-966.

However, the processes described in the above-listed publications are not efficient for scale-up since they require cryogenic temperatures, expensive starting materials, toxic reagents or many steps. The lack of an efficient process to manufacture 1-cyclopentylethanone is further also mirrored by the difficulty in sourcing this compound on kilogram scale for a reasonable price and delivery time.

Therefore, the purpose of the present invention is to provide a new, efficient and cost effective process for the preparation of 2-cyclopentyl-6-methoxy-isonicotinic acid, which is suitable for industrial scale synthesis.

### DESCRIPTION OF THE INVENTION

(i) The present invention relates to a process for the preparation of 1-cyclopentylethanone (3),

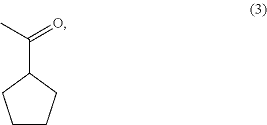

comprising the conversion of tert.-butyl 1-acetylcyclopentanecarboxylate (2)

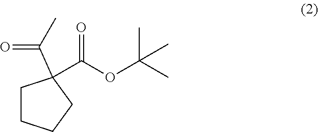

into 1-cyclopentylethanone (3) by means of acidic hydrolysis.
(ii) One embodiment of the present invention relates to a process according to embodiment (i), comprising the reaction of tert.-butyl acetoacetate (1) with 1,4-dibromobutane, to obtain tert.-butyl 1-acetylcyclopentanecarboxylate (2):

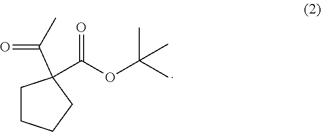

(iii) One embodiment of the present invention relates to a process according to embodiment (i) or (ii), wherein 1-cyclopentylethanone (3) is reacted with an alkyl oxalic acid ester ROC(O)C(O)OR to generate compound (4)

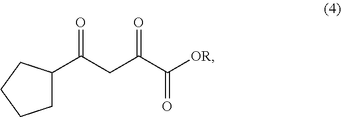

which is then reacted with cyanacetamide to generate compound (5),

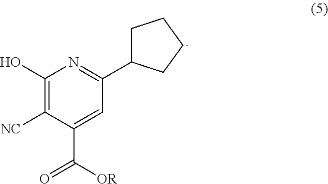

wherein R is ethyl, methyl, butyl, or tert-butyl.
(iv) In one embodiment, R is preferably ethyl.
(v) In one embodiment of the present invention, the process according to embodiment (iii) or (iv) further comprises the reaction of compound (5) with an aqueous acid to give 2-cyclopentyl-6-hydroxyisonicotinic acid (6)

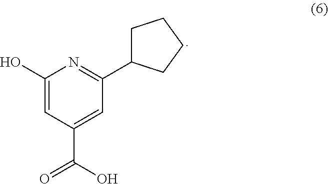 [SMILES: O=C(O)c1cc(O)nc(C2CCCC2)c1]

(vi) In one embodiment of the present invention, the process according to embodiment (v) further comprises the reaction of compound (6) with HC(OMe)₃ under acid catalysis to give methyl 2-cyclopentyl-6-hydroxyisonicotinate (7)

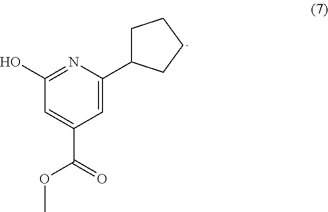 [SMILES: COC(=O)c1cc(O)nc(C2CCCC2)c1]

(vii) In one embodiment of the present invention, the process according to embodiment (vi) further comprises the reaction of compound (7) with a chlorination reagent to give methyl 2-chloro-6-cyclopentylisonicotinate (8)

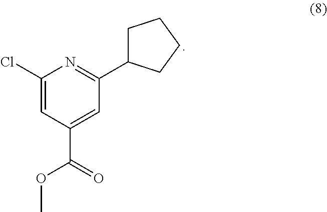 [SMILES: COC(=O)c1cc(Cl)nc(C2CCCC2)c1]

(viii) In one embodiment of the present invention, the process according to embodiment (v) further comprises the reaction of compound (6) with phosphorous oxychloride (POCl₃), followed by treatment with methanol, to give methyl 2-chloro-6-cyclopentylisonicotinate (8).
(ix) In one embodiment of the present invention, the process according to embodiment (vii) or (viii) further comprises the reaction of compound (8) with NaOMe/MeOH, followed by hydrolysis of the ester, to give 2-cyclopentyl-6-methoxy-isonicotinic acid (I):

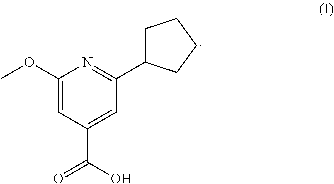 [SMILES: COc1cc(C(=O)O)cc(C2CCCC2)n1]

### DETAILED DESCRIPTION OF THE INVENTION

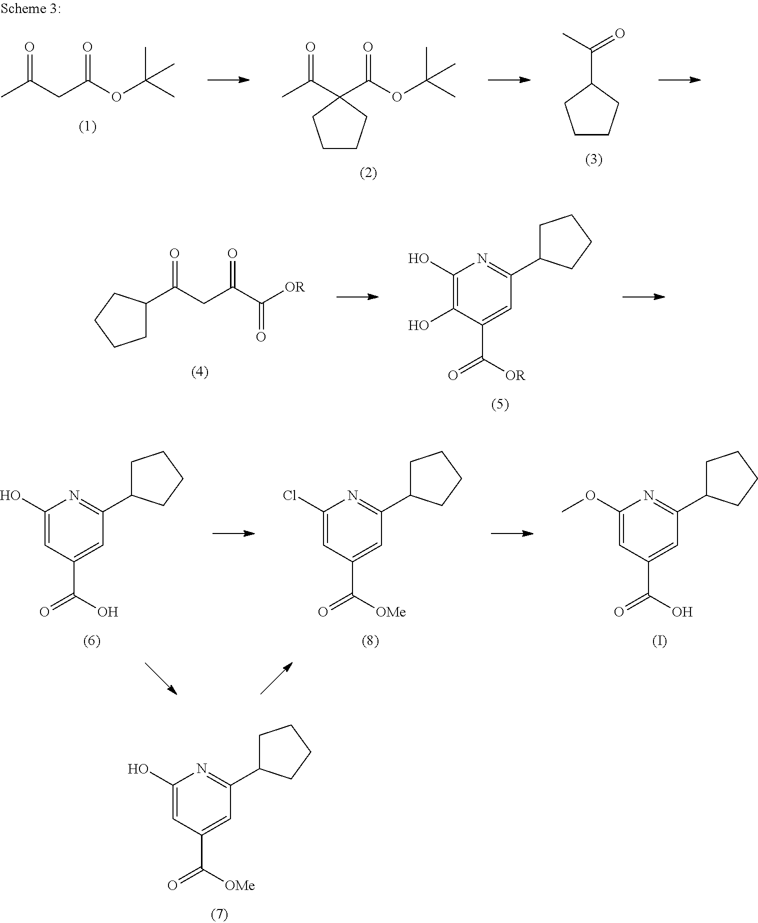

Embodiments (i) and (ii) as described above can be described in more detail: Tert.-butyl acetoacetate (1) is converted to tert.-butyl 1-acetylcyclopentanecarboxylate (2), by reacting compound (1) with 1,4-dibromobutane in aqueous base such as 20-60%, 25-55%, 25-50% or preferably 32-50% NaOH, most preferably 32% NaOH, in the presence of a phase transfer catalyst such as tetrabutylammonium bromide or iodide, preferably tetrabutylammonium bromide (TBABr). Alternatively, the base potassium carbonate or sodium carbonate in DMSO and in presence of a phase catalyst can be used as well (see Tetrahedron Letters 2005, 46, 635-638). Thereby, 2 to 2.5 equivalents of potassium carbonate are used.

The temperature of the mixture is kept at a temperature between 45-65° C., 45-60° C., 45-50° C., or preferably 50° C. When the system K₂CO₃/DMSO is used, a temperature of 20 to 30° C., preferably around 25° C. is sufficient.

1,4-Dibromobutane is added to the mixture in 1 equivalent, the phase transfer catalyst in a catalytic amount from 0.03 to 0.1 equivalents, preferably around 0.05 equivalents, and the alkyl acetoacetate is added from 0.8 to 1.2 equivalents, preferably 1 equivalent. The aqueous base is added in excess.

The reaction time is from 1 h to 10 h, from 2 h to 8 h, from 3 h to 7 h, from 4 h to 6 h, or preferably the reaction time is 5 hours. The system K₂CO₃/DMSO affords a longer reaction time, i.e. 15-25 h, preferably around 20 h.

After the reaction is completed, the organic layer is separated. Preferably, the organic layer is washed with aqueous acid, for example with 1N HCl. However, also other acids can be used.

In a second step, tert.-butyl 1-acetylcyclopentanecarboxylate (2) is converted to 1-cyclopentylethanone (3) by means of acidic hydrolysis. Thereby, tert.-butyl 1-acetylcyclopentanecarboxylate (2) is added to acid such as HCl, aqueous sulphuric acid, or trifluoroacetic acid (TFA).

In case of aqueous hydrochloric acid (HCl), concentrations of 25 to 32% HCl may be used, preferred is concentrated aq. HCl, i.e. 32% HCl. Alternatively, non-aqueous HCl solutions may be used as well, for instance 5M HCl in isopropanol. Preferably, 32% HCl is used.

In case of sulphuric acid, aqueous concentrations of 40-60%, 45-55% and preferably 50% may be used.

The reaction temperature can range from 50° C. to reflux. Preferably, the reaction temperature is kept at 60° C. to 80° C. in case of HCl and TFA, and around 120° C. for sulphuric acid.

The work up is done in usual way. Preferably, the mixture is washed with aqueous sodium chloride solution. It may be neutralized with a base before. After drying the organic layer, and filtration, the solvent is evaporated to give crude 1-cyclopentylethanone (3).

The conversion of compound (1) to 1-cyclopentylethanone (3) via compound (2) can be performed sequentially without specifically purifying compound (2).

Embodiment (iii) as described above can be described in more detail: 1-Cyclopentylethanone (3) is converted to the alkyl 4-cyclopentyl-2,4-dioxobutanoate (4) by reacting it with a dialkyl oxalate (dialkyl oxalic acid ester), in a solvent such as THF or Methyl THF in the presence of a base, such as KOtBu, NaOEt or NaOMe. Preferred are THF and KOtBu.

The base is added in an amount of 1 to 1.3 equivalents, preferred is 1.1 equivalent. 1-Cyclopentylethanone (3) is added in an amount of 1 equivalent, and the dialkyl oxalate is added in an amount of 0.8 to 1.2 equivalents, preferably 1 equivalent.

The person skilled in the art is well aware of the reaction conditions such as temperatures. The initial temperature range in the above reaction is from −23° C. to less than −18° C., and then kept at −23° C. to −5° C., from −20 to −10° C., or from −18° C. to −10° C. The initial temperature range is kept for a time depending on the scale of synthesis. For instance, it could be around 10 minutes to 1 hour. Afterwards, the reaction mixture is allowed to warm up to around 10 to 20° C., preferably around 15° C.

The work up of the reaction mixture in order to isolate the intermediate alkyl 4-cyclopentyl-2,4-dioxobutanoate (4) is known to the skilled person in the art (see US2008/242661 A1, 2008 or US2004/220186 A1, 2004). To the mixture is added aqueous acid such as HCl, for instance 2M HCl, and an organic solvent, for instance an ether such as TBME, the organic layer is separated and washed with water or aqueous salt solutions or buffers. The product is then isolated by evaporating the organic phase.

However, in order to convert 1-cyclopentylethanone (3) to alkyl 2-hydroxy-3-cyano-6-cyclopentyl-isonicotinate (5), the isolation of alkyl 4-cyclopentyl-2,4-dioxobutanoate (4) is not necessary. Rather, to the reaction mixture above, containing compound (4), cyano acetamide is added. The amount of cyano acetamide is between 1 to 1.4 equivalents, preferably 1.2 equivalents.

The reaction time may be from around 16 to around 25 hours, for instance around 20 hours. The reaction is performed at ambient temperature, for instance at 20 to 25° C., preferably at around 22° C.

To the reaction mixture is then added water, and the reaction mixture is concentrated by removing most of the solvent and water.

Oxalic acid esters ROC(O)C(O)OR may be selected from diethyl oxalate, dimethyl oxalate, dibutyl oxalate, or di-tert-butyl oxalate, preferred is diethyl oxalate.

Depending on the used oxalic acid ester ROC(O)C(O)OR, the following intermediates (4) are obtained:

ethyl 4-cyclopentyl-2,4-dioxobutanoate, which is preferred, methyl 4-cyclopentyl-2,4-dioxobutanoate, butyl 4-cyclopentyl-2,4-dioxobutanoate, or tert-butyl 4-cyclopentyl-2,4-dioxobutanoate.

Depending on compound (4), the following intermediates (5) are obtained:

ethyl 2-hydroxy-3-cyano-6-cyclopentyl-isonicotinate, which is preferred, methyl 2-hydroxy-3-cyano-6-cyclopentyl-isonicotinate, butyl 2-hydroxy-3-cyano-6-cyclopentyl-isonicotinate, or tert-butyl 2-hydroxy-3-cyano-6-cyclopentyl-isonicotinate.
(iv) In one embodiment, R is ethyl, i.e. compound (4) is ethyl 4-cyclopentyl-2,4-dioxobutanoate, and compound (5) is ethyl 2-hydroxy-3-cyano-6-cyclopentyl-isonicotinate.
(v) In one embodiment of the invention, the obtained alkyl 2-hydroxy-3-cyano-6-cyclopentyl-isonicotinate (5) is further converted to 2-cyclopentyl-6-hydroxyisonicotinic acid (6) with and aqueous acid, for example HCl at a concentration of 30% to 34%, preferably 32%.

The reaction is performed at a temperature ranging from 90 to 100° C., preferably at 100° C. The reaction time may be from around 20 to around 25 hours, for example around 22 hours. The work up is known by a person skilled in the art. For example around half of the solvent is removed and the obtained suspension is diluted with water and cooled to around 5° C. to 15° C., preferably 10° C., before being filtered to obtain 2-cyclopentyl-6-hydroxyisonicotinic acid (6). The conversion of Compound (3) to Compound (6) via Compound (4) and (5) can be performed sequentially without further purifying Compounds (4) and (5).

Embodiment (vi) as described above can be described in more detail: 2-Cyclopentyl-6-hydroxyisonicotinic acid (6) is converted to methyl 2-cyclopentyl-6-hydroxyisonicotinate (7), by reacting compound (6) with trimethylorthoformiate in methanol in the presence of a acid, such as sulphuric acid or with MeOH and sulphuric acid without trimethylorthoformiate.

2-Cyclopentyl-6-hydroxyisonicotinic acid (6) is added in an amount of 1 equivalent, trimethylorthoformiate is added in an amount of 1.9 to 2.2 equivalents, preferably 2 equivalents, and the acid is added in an amount of 1 to 1.4 equivalents. The solvent is added in excess.

The skilled person is aware of the temperature ranges and in particular of the reaction times. The reaction is preferably kept at reflux temperature, or a temperature in the range of 60 to 65° C. Also the work up is known to the skilled in the art. Preferably, the solvent is removed (under reduced pressure) and water is added to obtain a suspension containing the product, which can be isolated by filtration, preferably at a temperature below 15° C., preferably around 10° C.

In embodiment (vii), the obtained methyl 2-cyclopentyl-6-hydroxyisonicotinate (7) is converted to methyl 2-chloro-6-cyclopentylisonicotinate (8), by reacting Compound (7) with a chlorination reagent, for example with phenylphosphonic dichloride, phosphoryl chloride or thionyl chloride, and preferably with phenylphosphonic dichloride.

Methyl 2-cyclopentyl-6-hydroxyisonicotinate (7) is added in an amount of 1 equivalent, the chlorination reagent is added in an amount of 1.5 to 2.5 equivalents, depending also on the nature of the chlorination reagent. For example, the chlorination reagent is added in an amount of 2 equivalents.

The skilled person is aware of the temperature ranges and in particular of the reaction times to be taken.

The reaction temperature is kept at a range from 120 to 140° C., preferably at around 130° C. The work up is known to the person skilled in the art. Preferably, the reaction mixture is added to a mixture of an aqueous buffer and an organic solvent. For example, potassium phosphates in water and isopropyl acetate can be used. After phase separation, the organic fraction is collected and purified, for example by distillation.

Embodiment (viii) as Described Above can be Described in More Detail: 2-Cyclopentyl-6-hydroxyisonicotinic acid (6) is converted to methyl 2-chloro-6-cyclopentylisonicotinate (8), by reacting Compound 6 with phosphorous oxychloride (POCl₃), followed by treatment with methanol, to give compound (8).

2-Cyclopentyl-6-hydroxyisonicotinic acid (6) is added in an amount of 1 equivalent, phosphorous oxychloride (POCl₃) is added in an amount of 1.5 to 12 equivalents or 8 to 12 equivalents, preferably in an amount of around 10 equivalents.

The skilled person is aware of the temperature ranges and in particular of the reaction time to be taken.

The reaction temperature is kept at a range from 110 to 120° C., preferably around 115° C. The reaction time is from 3 to 5 h, preferably 4 h.

The reaction mixture is concentrated by distilling off the excess phosphorous oxychloride (POCl₃). An organic solvent can be added for diluting the concentrate and methanol is added in order to produce the methyl ester.

The workup is known to the person skilled in the art.

The mixture can be concentrated again, diluted with an organic solvent not miscible with water, and washing of the organic layer with water or aqueous salt or buffer solutions can follow. The organic layer is then ready for being concentrated again to give the product (8).

Embodiment (ix) as described above can be described in more detail: Methyl 2-chloro-6-cyclopentylisonicotinate (8) is reacted with NaOMe/MeOH, followed by hydrolysis of the ester, to give 2-cyclopentyl-6-methoxy-isonicotinic acid (I). Methyl 2-chloro-6-cyclopentylisonicotinate (8) is added in an amount of 1 equivalent, and sodium methanolate in methanol is added in excess, e.g. in a range of 8 equivalent to 15 equivalents, preferably around 10 equivalents.

The skilled person is aware of the temperature ranges and in particular of the reaction time to be taken.

The reaction temperature may be kept at reflux temperature. Reaction time may range from 10 to 48 hours.

Hydrolysis and workup are known to the person skilled in the art. Preferably, water is added to the reaction mixture and methanol is distilled off.

Then, the residue is acidified, for example to a pH of around 1 to 1.5, preferably to about 1. Aqueous hydrochloric acid may be used.

To the resulting mixture may then be added an organic solvent immiscible with water, so that the organic layer can be washed, separated and concentrated to obtain the product 2-cyclopentyl-6-methoxyisonicotinic acid (I).

Further purification methods are known in the art.

(x) The present invention further relates to a preferred intermediate of the process for the preparation of 2-cyclopentyl-6-methoxy-isonicotinic acid, which is ethyl 2-cyclopentyl-5-cyano-6-hydroxyisonicotinate (5).
(xi) The present invention further relates to a preferred intermediate of the process for the preparation of 2-cyclopentyl-6-methoxy-isonicotinic acid, which is 2-cyclopentyl-6-hydroxyisonicotinic acid (6).
(xii) The present invention further relates to a preferred intermediate of the process for the preparation of 2-cyclopentyl-6-methoxy-isonicotinic acid, which is methyl 2-cyclopentyl-6-hydroxyisonicotinate (7).
(xiii) The present invention further relates to a preferred intermediate of the process for the preparation of 2-cyclopentyl-6-methoxy-isonicotinic acid, which is methyl 2-chloro-6-cyclopentylisonicotinate (8).
(xiv) The present invention further relates to a process for the preparation of the pyridine-4-yl derivatives of formula (PD), wherein R^{a} is a cyclopentyl group, comprising the process according to any one of embodiments i) to ix).

The preparation of pyridine-4-yl derivatives of formula (PD), wherein R^{a} is cyclopentyl, from 2-cyclopentyl-6-methoxy-isonicotinic acid is described in detail in WO 2011/007324. In particular and as also described in WO 2011/007324, 5-pyridin-4-yl-[1,2,4]oxadiazole derivatives of formula (PD), wherein R^{a} is a cyclopentyl group, may be prepared by reacting a compound of Structure 9 in a solvent such as toluene, pyridine, DMF, THF, dioxane, DME, etc. at rt or elevated temperatures in the presence or absence of auxiliaries such as acids (e.g. TFA, acetic acid, HCl, etc.), bases (e.g. NaH, NaOAc, Na₂CO₃, K₂CO₃, NEt₃, etc.), tetraalkylammonium salts, or water removing agents (e.g. oxalyl chloride, a carboxylic acid anhydride, POCl₃, PCl₅, P₄O₁₀, molecular sieves, Burgess reagent, etc.) (Lit.: e.g. A. R. Gangloff, J. Litvak, E. J. Shelton, D. Sperandio, V. R. Wang, K. D. Rice, Tetrahedron Lett. 42 (2001), 1441-1443; T. Suzuki, K. Iwaoka, N. Imanishi, Y. Nagakura, K. Miyta, H. Nakahara, M. Ohta, T. Mase, Chem. Pharm. Bull. 47 (1999), 120-122; R. F. Poulain, A. L. Tartar, B. P. Deprez, Tetrahedron Lett. 42 (2001), 1495-1498; R. M. Srivastava, F. J. S. Oliveira, D. S. Machado, R. M. Souto-Maior, Synthetic Commun. 29 (1999), 1437-1450; E. 0. John, J. M. Shreeve, Inorganic Chemistry 27 (1988), 3100-3104; B. Kaboudin, K. Navaee, Heterocycles 60 (2003), 2287-2292).

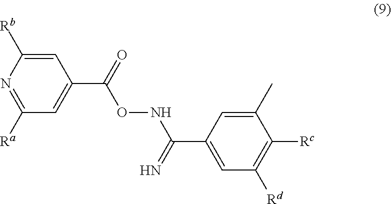

(wherein R^{a} is cyclopentyl and R^{b}, R^{c} and R^{d} are as defined above.)

Compounds of Structure 9 may be prepared by reacting 2-cyclopentyl-6-methoxy-isonicotinic acid with a compound of Structure 10 in a solvent such as DMF, THF, DCM, etc. in the presence of one or more coupling agents such as TBTU, DCC, EDC, HBTU, CDI, etc. and in the presence or absence of a base such as NEt₃, DIPEA, NaH, K₂CO₃, etc. (Lit.: e.g. A. Hamze, J.-F. Hernandez, P. Fulcrand, J. Martinez, J. Org. Chem. 68 (2003) 7316-7321).

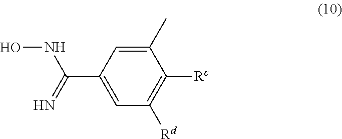

(wherein R^{c} and R^{d} are as defined above.)

The preparation of compounds of Structure 10 is also described in WO 2011/007324.

Pyridine-4-yl derivatives of formula (PD), which are readily prepared by using 2-cyclopentyl-6-methoxy-isonicotinic acid, include:
(S)-3-{4-[5-(2-cyclopentyl-6-methoxy-pyridin-4-yl)-[1,2,4]oxadiazol-3-yl]-2-ethyl-6-me thyl-phenoxy}-propane-1,2-diol;
(R)-3-{4-[5-(2-cyclopentyl-6-methoxy-pyridin-4-yl)-[1,2,4]oxadiazol-3-yl]-2-ethyl-6-m ethyl-phenoxy}-propane-1,2-diol;
ethanesulfonic acid {2-chloro-4-[5-(2-cyclopentyl-6-methoxy-pyridin-4-yl)-[1,2,4]-oxadiazol-3-yl]-6-methyl-phenyl}amide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-acetamide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-N-methyl-acetamide;
N-{2-chloro-4-[5-(2-cyclopentyl-6-methoxy-pyridin-4-yl)-[1,2,4]oxadiazol-3-yl]-6-meth yl-phenyl}-methanesulfonamide;
(S)-3-{2-chloro-4-[5-(2-cyclopentyl-6-methoxy-pyridin-4-yl)-[1,2,4]oxadiazol-3-yl]-6-methyl-phenoxy}-propane-1,2-diol;
N—((S)-3-{2-chloro-4-[5-(2-cyclopentyl-6-methoxy-pyridin-4-yl)-[1,2,4]oxadiazol-3-yl]-6-methyl-phenoxy}-2-hydroxy-propyl)-2-hydroxy-acetamide;
N-{4-[5-(2-cyclopentyl-6-methoxy-pyridin-4-yl)-[1,2,4]oxadiazol-3-yl]-2-ethyl-6-methyl-phenyl}methanesulfonamide;
(S)-3-{4-[3-(2-cyclopentyl-6-methoxy-pyridin-4-yl)-[1,2,4]oxadiazol-5-yl]-2-ethyl-6-me thyl-phenoxy}-propane-1,2-diol;
(R)-3-{4-[3-(2-cyclopentyl-6-methoxy-pyridin-4-yl)-[1,2,4]oxadiazol-5-yl]-2-ethyl-6-m ethyl-phenoxy}-propane-1,2-diol;
N—((S)-3-{4-[3-(2-cyclopentyl-6-methoxy-pyridin-4-yl)-[1,2,4]oxadiazol-5-yl]-2-ethyl-6-methyl-phenoxy}-2-hydroxy-propyl)-2-hydroxy-acetamide;
N—((R)-3-{4-[3-(2-cyclopentyl-6-methoxy-pyridin-4-yl)-[1,2,4]oxadiazol-5-yl]-2-ethyl-6-methyl-phenoxy}-2-hydroxy-propyl)-2-hydroxy-acetamide;
(S)-3-{4-[5-(2-cyclopentyl-6-methoxy-pyridin-4-yl)-[1,3,4]oxadiazol-2-yl]-2-ethyl-6-me thyl-phenoxy}-propane-1,2-diol;
(R)-3-{4-[5-(2-cyclopentyl-6-methoxy-pyridin-4-yl)-[1,3,4]oxadiazol-2-yl]-2-ethyl-6-m ethyl-phenoxy}-propane-1,2-diol;
N—((S)-3-{4-[5-(2-cyclopentyl-6-methoxy-pyridin-4-yl)-[1,3,4]oxadiazol-2-yl]-2-ethyl-6-methyl-phenoxy}-2-hydroxy-propyl)-2-hydroxy-acetamide; and
N—((R)-3-{4-[5-(2-cyclopentyl-6-methoxy-pyridin-4-yl)-[1,3,4]oxadiazol-2-yl]-2-ethyl-6-methyl-phenoxy}-2-hydroxy-propyl)-2-hydroxy-acetamide.

Preferred pyridine-4-yl derivatives of formula (PD), which are readily prepared by using 2-cyclopentyl-6-methoxy-isonicotinic acid, include:
(S)-3-{4-[5-(2-cyclopentyl-6-methoxy-pyridin-4-yl)-[1,2,4]oxadiazol-3-yl]-2-ethyl-6-me thyl-phenoxy}-propane-1,2-diol;
(R)-3-{4-[5-(2-cyclopentyl-6-methoxy-pyridin-4-yl)-[1,2,4]oxadiazol-3-yl]-2-ethyl-6-m ethyl-phenoxy}-propane-1,2-diol;
ethanesulfonic acid {2-chloro-4-[5-(2-cyclopentyl-6-methoxy-pyridin-4-yl)-[1,2,4]-oxadiazol-3-yl]-6-methyl-phenyl}amide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-acetamide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-N-methyl-acetamide;
N-{2-chloro-4-[5-(2-cyclopentyl-6-methoxy-pyridin-4-yl)-[1,2,4]oxadiazol-3-yl]-6-meth yl-phenyl}-methanesulfonamide;
(S)-3-{2-chloro-4-[5-(2-cyclopentyl-6-methoxy-pyridin-4-yl)-[1,2,4]oxadiazol-3-yl]-6-methyl-phenoxy}-propane-1,2-diol;
N—((S)-3-{2-chloro-4-[5-(2-cyclopentyl-6-methoxy-pyridin-4-yl)-[1,2,4]oxadiazol-3-yl]-6-methyl-phenoxy}-2-hydroxy-propyl)-2-hydroxy-acetamide; and
N-{4-[5-(2-cyclopentyl-6-methoxy-pyridin-4-yl)-[1,2,4]oxadiazol-3-yl]-2-ethyl-6-methyl-phenyl}-methanesulfonamide.

2-Cyclopentyl-6-methoxy-isonicotinic acid is especially suitable for the preparation of 5-pyridin-4-yl-[1,2,4]oxadiazole derivatives of formula (PD), wherein R^{a} is a cyclopentyl group.

Any reference hereinbefore or hereinafter to a compound is to be understood as referring also to salts, especially pharmaceutically acceptable salts, of such compound, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

As already mentioned above, the purpose of the present invention is to provide a new, efficient and cost effective process for the preparation of 2-cyclopentyl-6-methoxy-isonicotinic acid, which is suitable for industrial scale synthesis. Even though the process to prepare 1-cyclopentylethanone (3) is a key step for this purpose, also as described in particular in embodiment (i) above, it is still to be said that

(a) The present invention relates to a process for the preparation of 2-cyclopentyl-6-methoxy-isonicotinic acid,

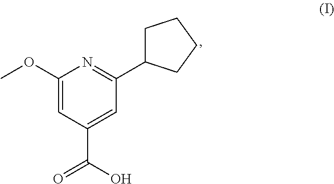 [SMILES: COc1cc(C(=O)O)cc(C2CCCC2)n1]

comprising a reaction sequence a) to produce 1-cyclopentylethanone (3) in which tert.-butyl acetoacetate (1) is reacted with 1,4-dibromobutane, to obtain tert.-butyl 1-acetylcyclopentanecarboxylate (2), and converting it by means of acidic hydrolysis to 1-cyclopentylethanone (3):

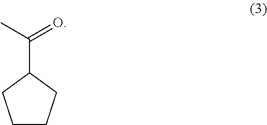

(b) One embodiment of the present invention relates to a process according to embodiment (a), comprising a reaction sequence b) of converting 1-cyclopentylethanone (3) to 2-cyclopentyl-6-hydroxyisonicotinic acid (6):

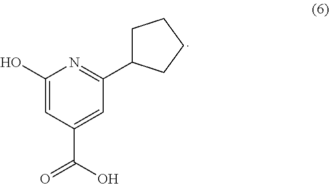 [SMILES: O=C(O)c1cc(O)nc(C2CCCC2)c1]

(c) One embodiment of the present invention relates to a process according to embodiment (b), wherein in reaction sequence b), 1-cyclopentylethanone (3) is reacted with an alkyl oxalic acid ester ROC(O)C(O)OR to generate compound (4)

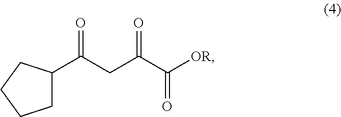

which is then reacted with cyanacetamide to generate compound (5),

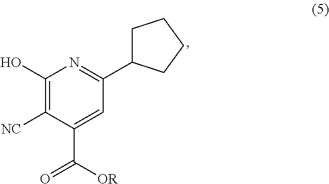

wherein R is ethyl, methyl, butyl, or tert-butyl,
followed by treating compound (5) with an aqueous acid to give 2-cyclopentyl-6-hydroxyisonicotinic acid (6).
(d) In one embodiment, R is preferably ethyl.
(e) Another embodiment of present invention relates to a process according to embodiment (b), (c), or (d) wherein 2-cyclopentyl-6-hydroxyisonicotinic acid (6) is converted to methyl 2-chloro-6-cyclopentylisonicotinate (8):

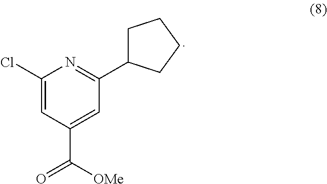 [SMILES: COC(=O)c1cc(Cl)nc(C2CCCC2)c1]

(f) One embodiment of present invention relates to a process according to embodiment (e), wherein 2-cyclopentyl-6-hydroxyisonicotinic acid (6) is reacted with HC(OMe)₃ under acid catalysis to give methyl 2-cyclopentyl-6-hydroxyisonicotinate (7)

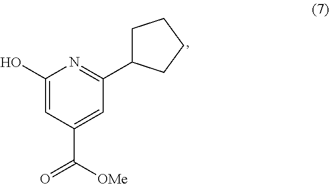 [SMILES: COC(=O)c1cc(O)nc(C2CCCC2)c1]

which is then reacted with a chlorination reagent to give methyl 2-chloro-6-cyclopentylisonicotinate (8).
(g) One embodiment of present invention relates to a process according to embodiment (e), wherein 2-cyclopentyl-6-hydroxyisonicotinic acid (6) is reacted with phosphorous oxychloride (POCl₃), followed by treatment with methanol, to give compound (8).
(h) One embodiment of present invention relates to a process according to embodiment (e), (f) or (g), wherein methyl 2-chloro-6-cyclopentylisonicotinate (8) is reacted with NaOMe/MeOH, followed by hydrolysis of the ester, to give 2-cyclopentyl-6-methoxy-isonicotinic acid (I):

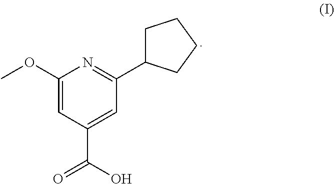 [SMILES: COc1cc(C(=O)O)cc(C2CCCC2)n1]

(j) One preferred embodiment of present invention relates to a process for the preparation of 1-cyclopentylethanone (3):

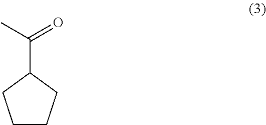

comprising the conversion of tert.-butyl 1-acetylcyclopentanecarboxylate (2)

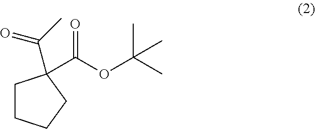

into 1-cyclopentylethanone (3) by means of acidic hydrolysis.
(k) Another specific embodiment of present invention relates to a process according to embodiment (j), comprising the reaction of tert.-butyl acetoacetate (1) with 1,4-dibromobutane, to obtain tert.-butyl 1-acetylcyclopentanecarboxylate (2):

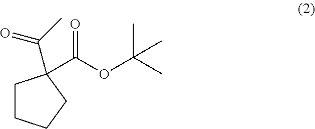

The general and specific process conditions described herein above and herein below also apply to the processes of embodiments (a)-(k).

### Examples

The following examples illustrate the invention but do not at all limit the scope thereof.

All temperatures given are external temperatures and are stated in ° C. Compounds are characterized by ¹H-NMR (400 MHz) or ¹³C-NMR (100 MHz) (Bruker; chemical shifts are given in ppm relative to the solvent used; multiplicities: s=singlet, d=doublet, t=triplet, p=pentuplet, hex=hexet, hept=heptet, m=multiplet, br=broad, coupling constants are given in Hz), internal standard for quantitative NMR was 1,4-dimethoxybenzene; by LC-MS (Agilent MS detector G1956B with Agilent 1200 Binary Pump and DAD), t_{R} is given in minutes; or by GC-MS (Thermo Scientific, Trace Ultra, DSQ II detector), t_{R} is given in minutes.

GC-MS method:

| | |
|---|---|
| Injection | 1.00 μL |
| volume: | |
| Column: | Zebron ZB-5-MS, 15 m × 0.25 mm ID, 0.25 μm film |
| Column | 2.0 mL/min |
| flow: | |
| Carrier gas: | Helium |
| Split ratio: | 20 |
| Split- | 200° C. |
| splitless | |
| inlet | |
| temperature: | |
| Temperature | 60-300° C. from 0 to 4.0 min, 300° C. isotherm from 4.0 to |
| gradient: | 5.0 min |
| Ionization: | Chemical ionization with CH₄ as reagent gas |

LC-MS method:

| Injection volume: | 5 μl | |
|---|---|---|
| Column: | Kinetex C18, 2.6 μm, 2.1 × 50 mm | |
| Column flow: | 1 mL/min | |
| Eluent: | Eluent A: Water, 0.08% TFA | |
| | Eluent B: Acetonitrile | |
| Gradient: | 2.0 min | 95% B |
| | 2.8 min | 95% B |
| | 3.0 min | 5% B |

| | |
|---|---|
| Temperature: | 40° C. |

### ABBREVIATIONS (AS USED HEREIN)

aq. aqueous
b.p. boiling point
Burgess reagent methoxycarbonylsulfamoyl triethylammonium hydroxide
DCM dichloromethane
CDI carbonyl diimidazole
DCC N,N′-dicyclohexyl carbodiimide
DIPEA Huning's base, diethylisopropylamine
DME 1,2-dimethoxyethane
DMF dimethylformamide
DMSO dimethylsulfoxide
EDC N-(3-dimethylaminopropyl)-N′-ethyl-carbodiimide
eq. equivalent(s)
Et ethyl
GC-MS gas chromatography—mass spectrometry
h hour(s)
HBTU O-(benzotriazol-1-yl)-N,N,N′,N′-tetramethyluronium hexafluorophosphate
KOtBu potassium tert.-butylate
LC-MS liquid chromatography—mass spectrometry
Lit. literature
Me methyl
MeOH methanol
min minute(s)
m.p. melting point
NaOAc sodium acetate
NMR nuclear magnetic resonance
org. organic
TBABr tetrabutylammonium bromide
TBME tert.-butyl methyl ether
TBTU 2-(1H-benzotriazole-1-yl)-1,2,3,3-tetramethyluronium tetrafluoroborate
TFA trifluoroacetic acid
THF tetrahydrofuran
t_{R} retention time
% a/a area % (purity by area %)

### EXAMPLES

### Example 1a

### 1-Cyclopentylethanone

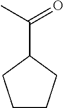

A mixture of 1,4 dibromobutane (273 g, 1 eq.), tetrabutylammonium bromide (20 g, 0.05 eq.) in 32% NaOH (1 L) was heated to 50° C. Tert.-butyl acetoacetate (200 g, 1 eq.) was added keeping the maximum internal temperature below 55° C. The mixture was stirred for 5 h at 50° C. The stirrer was stopped and the org. layer was separated. The org. layer was washed with 1N HCl (500 mL). The org. layer was added to 32% HCl (300 mL) at an external temperature of 60° C. The mixture was stirred at 60° C. for 3.5 h and cooled to 40° C. The mixture was washed with brine (60 mL). The org. layer was washed with brine (150 mL) and dried with magnesium sulphate (8 g). The mixture was filtered and the product was purified by distillation (distillation conditions: external temperature: 70° C., head temperature: 40-55° C., pressure: 30-7 mbar) to obtain a colourless liquid; yield: 107 g (75%). Purity (GC-MS): 99.8% a/a; GC-MS: t_{R}=1.19 min, [M+1]⁺=113. ¹H NMR (CDCl₃): δ=2.86 (m, 1H), 2.15 (s, 3H), 1.68 (m, 8H).

### Example 1 b

### 1-Cyclopentylethanone

Tert-butyl 1-acetylcyclopentanecarboxylate (723 g, 3.41 mol) was added to 32% HCl (870 mL) at an internal temperature of 80° C. over a period of 2 h. The mixture was stirred at 80° C. for 1 h and cooled to 50° C. The stirrer was stopped and the org. layer was separated. The org. layer was washed with water (250 mL) and dried with magnesium sulphate (24 g). The mixture was filtered and the product was purified by distillation to obtain a colourless liquid; yield: 333.6 g (87%). Purity (GC-MS): 97.3% a/a; GC-MS: t_{R}=1.19 min, [M+1]⁺=113.

### Example 1c

### 1-Cyclopentylethanone

Tert-butyl 1-acetylcyclopentanecarboxylate (300 g, 1.41 mol) was added to 5 M HCl in isopropanol (600 mL) at an internal temperature of 60° C. over a period of 25 min. The mixture was stirred at 60° C. for 18 h and cooled to 20° C. Water (1 L) was added, the stirrer was stopped and the org. layer was separated. The org. layer was washed with water (500 mL). The crude product was purified by distillation to obtain a colourless liquid; yield: 115 g (72%). Purity (GC-MS): 87.2% a/a; GC-MS: t_{R}=1.19 min, [M+1]⁺=113.

### Example 1d

### 1-Cyclopentylethanone

Tert-butyl 1-acetylcyclopentanecarboxylate (514 g, 2.42 mol) was added to TFA (390 mL) at an internal temperature of 60° C. More TFA (200 mL) was added and the temperature was adjusted to 65° C. The mixture was stirred at 65° C. for 1 h. The reaction mixture was concentrated at 45° C. and 20 mbar. The residue was added to TBME (500 mL), ice (200 g) and 32% NaOH (300 mL). The aq. layer was separated and extracted with TBME (500 mL). The combined org. layers were concentrated to dryness to yield the crude 1-cyclopentylethanone. The crude product was purified by distillation to yield a colorless liquid: 221.8 g (82%). Purity (GC-MS): 90.2% a/a; GC-MS: t_{R}=1.19 min, [M+l]⁺=113.

### Example 1e

### 1-Cyclopentylethanone

Tert-butyl 1-acetylcyclopentanecarboxylate (534 g, 2.52 mol) was added to 50% H₂SO₄ (300 mL) at an internal temperature of 100° C. over a period of 40 min. The mixture was stirred at 120° C. for 2 h and cooled to 20° C. The stirrer was stopped and the org. layer was separated. The org. layer was washed with saturated NaHCO₃ solution (250 mL). The crude product was purified by distillation to obtain a colourless liquid; yield: 177 g (63%). Purity (GC-MS): 99.9% a/a; GC-MS: t_{R}=1.19 min, [M+1]+=113.

### Example 1f

### Tert-butyl 1-acetylcyclopentanecarboxylate

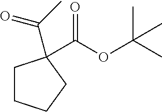

To a mixture of potassium carbonate (1 kg, 7.24 mol) and tetrabutylammonium iodide (10 g, 0.027 mol) in DMSO (3 L) was added a mixture of 1,4-dibromobutane (700 g, 3.24 mol) and tert.-butyl acetoacetate (500 g, 3.16 mol). The mixture was stirred at 25° C. for 20 h. To the reaction mixture was added water (4 L) and TBME (3 L). The mixture was stirred until all solids dissolved. The TBME layer was separated and washed with water (3×1 L). The org. layer was concentrated and the crude product was purified by distillation (distillation conditions: external temperature: 135° C., head temperature: 105-115° C., pressure: 25-10 mbar) to obtain a colourless liquid; yield: 537.6 g (80%). Purity (GC-MS): 90.5% a/a; GC-MS:

t_{R}=1.89 min, [M+1]⁺=213. ¹H NMR (CDCl₃): δ=2.16 (s, 3H), 2.06 (m, 4H), 1.63 (m, 4H), 1.45 (s, 9H).

### Example 1 g

### Tert-butyl 1-acetylcyclopentanecarboxylate

A mixture of 1,4 dibromobutane (281 g, 1 eq.) and tetrabutylammonium bromide (15 g, 0.05 eq.) in 50% NaOH (1 L) was heated to 50° C. Tert.-butyl acetoacetate (206 g, 1 eq.) was added keeping the maximum internal temperature below 55° C. The mixture was stirred for 5 h at 50° C. The stirrer was stopped and the org. layer was separated. The org. layer was washed with 1N HCl (500 mL). The crude product was purified by distillation to obtain a colourless liquid; yield: 199 g (72%). Purity (GC-MS): 97.8% a/a; GC-MS: t_{R}=1.89 min, [M+1]⁺=213.

### Example 2

### 2-Cyclopentyl-6-hydroxyisonicotinic acid

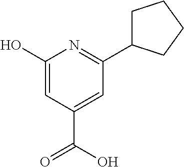 [SMILES: O=C(O)c1cc(O)nc(C2CCCC2)c1]

A 10 L reactor was charged with potassium tert.-butylate (220 g, 1.1 eq.) and THF (3 L). The solution was cooled to −20° C. A mixture of diethyloxalate (260 g, 1 eq.) and 1-cyclopentylethanone (200 g, 1.78 mol, 1 eq.) was added at a temperature below −18° C. The reaction mixture was stirred at −10° C. for 30 min and then warmed to 15° C. To the mixture was added cyano acetamide (180 g, 1.2 eq.). The mixture was stirred for 20 h at 22° C. Water (600 mL) was added and the reaction mixture was concentrated at 60° C. under reduced pressure on a rotary evaporator. 3.4 L solvent were removed. The reactor was charged with 32% HCl (5 L) and heated to 50° C. The residue was added to the HCl solution at a temperature between 44 and 70° C. The mixture was heated to 100° C. for 22 h. 2.7 L solvent were removed at 135° C. external temperature and a pressure of ca. 400 mbar. The suspension was diluted with water (2.5 L) and cooled to 10° C. The suspension was filtered. The product cake was washed with water (2.5 L) and acetone (3 L). The product was dried to obtain an off white solid; yield: 255 g (69%); purity (LC-MS): 100% a/a; LC-MS: t_{R}=0.964 min, [M+1]⁺=208; ¹H NMR (deutero DMSO): δ=12.67 (br, 2H), 6.63 (s, 1H), 6.38 (s, 1H), 2.89 (m, 1H), 1.98 (m, 2H), 1.63 (m, 6H).

### Example 3

### Methyl 2-cyclopentyl-6-hydroxyisonicotinate

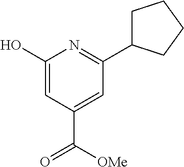 [SMILES: COC(=O)c1cc(O)nc(C2CCCC2)c1]

2-Cyclopentyl-6-hydroxyisonicotinic acid (1520.5 g, 7.3 mol, 1 eq.), methanol (15.2 L), trimethylorthoformiate (1.56 L, 2 eq.) and sulphuric acid (471 mL, 1.2 eq.) were mixed at 20° C. and heated to reflux for 18 h. 10 L solvent were removed at 95° C. external temperature and a pressure of ca. 800 mbar.

The mixture was cooled to 20° C. and added to water (7.6 L) at 50° C. The suspension was diluted with water (3.8 L), cooled to 10° C. and filtered. The cake was washed with water (3.8 L). The product was dried to obtain a yellowish solid; yield: 1568 g (97%); purity (LC-MS): 100% a/a; LC-MS: t_{R}=1.158 min, [M+1]⁺=222; ¹H NMR (deutero DMSO) δ=11.98 (br, 1H), 6.63 (m, 1H), 6.39 (s, 1H), 3.83 (s, 3H), 2.91 (m, 1H), 1.99 (m, 2H), 1.72 (m, 2H), 1.58 (m, 4H).

### Example 4a

### Methyl 2-chloro-6-cyclopentylisonicotinate

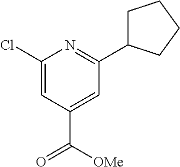 [SMILES: COC(=O)c1cc(Cl)nc(C2CCCC2)c1]

Methyl 2-cyclopentyl-6-hydroxyisonicotinate (50 g, 0.226 mol, 1 eq.) and phenylphosphonic dichloride (70 mL, 2 eq.) were heated to 130° C. for 3 h. The reaction mixture was added to a solution of potassium phosphate (300 g) in water (600 mL) and isopropyl acetate (600 mL) at 0° C. The mixture was filtered over kieselguhr (i.e. diatomite, Celite™) (50 g). The aq. layer was separated and discarded. The org. layer was washed with water (500 mL). The org. layer was concentrated to dryness at 65° C. and reduced pressure to obtain a black oil; yield: 50.4 g (93%); purity (LC-MS): 94% a/a.

The crude oil was purified by distillation at an external temperature of 130° C., head temperature of 106° C. and oil pump vacuum to yield a colourless oil; yield: 45.6 g (84%); purity (LC-MS): 100% a/a; LC-MS: t_{R}=1.808 min, [M+1]⁺=240; ¹H NMR (CDCl₃) δ=7.69 (s, 1H), 7.67 (s, 1H), 3.97 (s, 3H), 3.23 (m, 1H), 2.12 (m, 2H), 1.80 (m, 6H).

### Example 4b

### Methyl 2-chloro-6-cyclopentylisonicotinate

2-Cyclopentyl-6-hydroxyisonicotinic acid (147 g, 0.709 mol, 1 eq.) and phosphorous oxychloride (647 mL, 10 eq.) were heated to 115° C. for 4 h. The mixture was concentrated at normal pressure and an external temperature of 130-150° C. At 20° C. DCM (250 mL) was added. The solution was added to MeOH (1000 mL) below 60° C. The mixture was concentrated under reduced pressure at 50° C. DCM (1 L) was added to the residue and the mixture was washed with water (2×500 mL). The org. layer was concentrated to dryness under reduced pressure at 50° C. to obtain a black oil; yield: 181.7 g (107%); purity (LC-MS): 97% a/a. The product was contaminated with trimethyl phosphate.

### Example 5

### 2-Cyclopentyl-6-methoxyisonicotinic acid

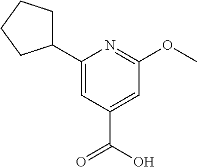 [SMILES: COc1cc(C(=O)O)cc(C2CCCC2)n1]

Methyl 2-chloro-6-cyclopentylisonicotinate (40 g, 0.168 mol, 1 eq.) and 5.4 M NaOMe in MeOH (320 mL, 10 eq.) were heated to reflux for 16 h. Water (250 mL) was added carefully at 80° C. external temperature. Methanol was distilled off at 60° C. and reduced pressure (300 mbar). The residue was acidified with 32% HCl (150 mL) and the pH was adjusted to 1. The mixture was extracted with isopropyl acetate (300 mL). The aq. layer was discarded. The org. layer was washed with water (200 mL). The org. solution was concentrated to dryness under reduced pressure at 60° C. to obtain a white solid; yield: 35.25 g (95%). The crude product was crystallized from acetonitrile (170 mL) to obtain a white solid; 31 g (84%); purity (LC-MS): 97.5% a/a.

LC-MS: t_{R}=1.516 min, [M+1]⁺=222; ¹H NMR (deutero DMSO) δ=13.50 (br s, 1H), 7.25 (s, 1H), 6.98 (s, 1H), 3.88 (s, 3H), 3.18 (m, 1H), 2.01 (m, 2H), 1.72 (m, 6H).

### Example 6

### Ethyl 4-cyclopentyl-2,4-dioxobutanoate

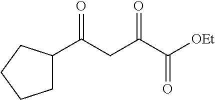 [SMILES: CCOC(=O)C(=O)CC(=O)C1CCCC1]

A solution of 20% potassium tert-butoxide in THF (595 mL, 1.1 eq.) and THF (400 mL) was cooled to −20° C. A mixture of diethyloxalate (130 g, 1 eq.) and 1-cyclopentylethanone (100 g, 0.891 mol, 1 eq.) was added at a temperature below −18° C. The reaction mixture was stirred at −10° C. for 30 min and then warmed to 15° C. To the mixture was added 2 M HCl (1 L) and TBME (1 L). The org. layer was separated and washed with water (1 L). The org. layer was evaporated to dryness on a rotary evaporator to obtain an oil; yield: 171 g (91%); purity (GC-MS): 97% a/a; GC-MS: t_{R}=2.50 min, [M+1]⁺=213; ¹H NMR δ: 14.55 (m, 1H), 6.41 (s, 1H), 4.37 (q, J=7.1 Hz, 2H), 2.91 (m, 1H), 1.79 (m, 8H), 1.40 (t, J=7.1 Hz, 3H).

### Example 7

### Ethyl 3-cyano-6-cyclopentyl-2-hydroxyisonicotinate

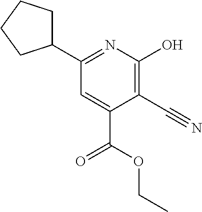 [SMILES: CCOC(=O)c1cc(C2CCCC2)nc(O)c1C#N]

Triethylamine (112 mL, 1 eq.) and cyanoacetamide (67.9 g, 1 eq.) was heated in ethanol to 65° C. Ethyl 4-cyclopentyl-2,4-dioxobutanoate (171 g, 0.807 mol, 1 eq.) was added to the mixture at 65° C. The mixture was stirred for 3 h at 65° C. The mixture was cooled to 20° C. and filtered. The product was washed with TBME (2×200 mL).

The product was dried to obtain a yellow solid; yield: 85 g (40%); purity (LC-MS): 97% a/a; LC-MS: t_{R}=1.41 min, [M+1]⁺=261; ¹H NMR (CDCl₃) δ: 12.94 (m, 1H), 6.70 (s, 1H), 4.50 (q, J=7.1 Hz, 2H), 3.11 (m, 1H), 2.21 (m, 2H), 1.96 (m, 2H), 1.78 (m, 4H), 1.48 (t, 3H).

### REFERENTIAL EXAMPLES

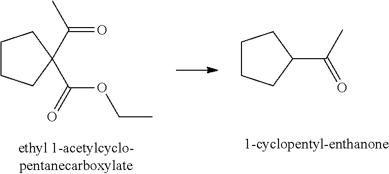

Original process described by Goldsworthy in J. Chem. Soc. 1934, 377-378.

According to Goldsworthy the ketonic ester (ethyl 1-acetylcyclopentanecarboxylate) (19.5 g) was refluxed for 24 h with a considerable excess of potash (19 g) in alcohol (150 cc), two-thirds of the alcohol then distilled off, the residue refluxed for 3 h, the bulk of the alcohol finally removed, saturated brine added, and the ketone extracted with ether. The oil obtained from the extract distilled at 150-160°/760 mm and yielded nearly 4 g of a colourless oil, b.p. 153-155°/760 mm, on redistillation. The semicarbazone, prepared from the ketone and a slight excess of equivalent amounts of semicarbazide and sodium acetate in saturated solution, alcohol just sufficient to clear the solution being finally added, rapidly separated; m.p. 145° after recrystallisation from acetone (Found: N, 24.5. C8H15ON3 requires N, 24.8%).

The process described by Goldsworthy has been reproduced using K₂CO₃ in the absence (Referential Example 1) and presence (Referential Example 2) of water.

### Referential Example 1

Ethyl 1-acetylcyclopentanecarboxylate (19.5 g, 0.106 mol) was refluxed for 24 h with K₂CO₃ (19 g, 0.137 mol, Aldrich: 347825) in ethanol (150 mL). GC-MS indicated a conversion to 3% of the desired product. The solvent was removed and the residue was extracted with ether and brine. Evaporation of solvent yielded 28.5 g of a yellow oil. GC-MS indicated ca. 86% a/a starting material, 3% a/a product.

### Referential Example 2

Ethyl 1-acetylcyclopentanecarboxylate (19.5 g, 0.106 mol) was refluxed for 24 h with K₂CO₃ (19 g, 0.137 mol, Aldrich: 347825) in ethanol (150 mL) in the presence of water (1.91 g, 1 eq.). GC-MS indicated a conversion to 17% of the desired product. The reaction mixture was discarded.

1. A process for the preparation of 1-cyclopentylethanone (3), 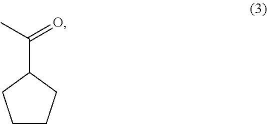
comprising the conversion of tert.-butyl 1-acetylcyclopentanecarboxylate (2) 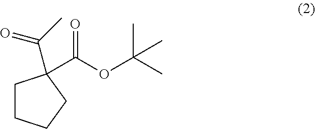
into 1-cyclopentylethanone (3) by means of acidic hydrolysis.

2. The process according to claim 1, comprising the reaction of tert.-butyl acetoacetate with 1,4-dibromobutane, to obtain tert.-butyl 1-acetylcyclopentanecarboxylate (2):

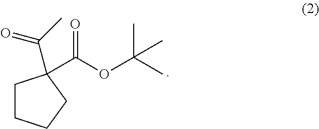

3. The process according to claim 1, further comprising the reaction of 1-cyclopentylethanone (3) with an alkyl oxalic acid ester ROC(O)C(O)OR to generate compound (4) 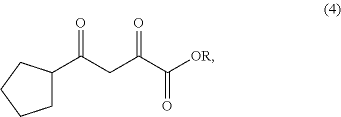
which is then reacted with cyanacetamide to generate compound (5), 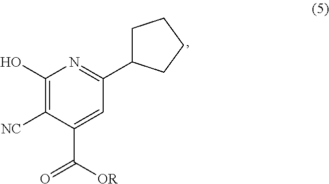
wherein R is ethyl, methyl, butyl, or tert-butyl.

4. The process according to claim 3, wherein R is ethyl.

5. The process according to claim 3, further comprising the reaction of compound (5) with an aqueous acid to give 2-cyclopentyl-6-hydroxyisonicotinic acid (6)

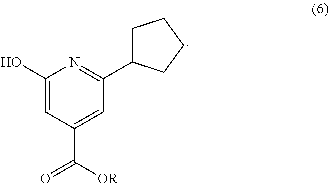

6. The process according to claim 5, further comprising the reaction of compound (6) with HC(OMe)₃ under acid catalysis to give methyl 2-cyclopentyl-6-hydroxyisonicotinate (7)

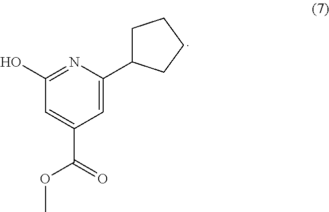 [SMILES: COC(=O)c1cc(O)nc(C2CCCC2)c1]

7. The process according to claim 6, further comprising the reaction of compound (7) with a chlorination reagent to give methyl 2-chloro-6-cyclopentylisonicotinate (8)

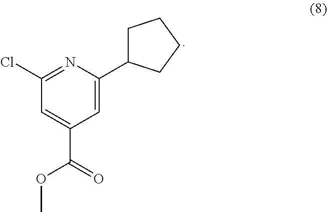 [SMILES: COC(=O)c1cc(Cl)nc(C2CCCC2)c1]

8. The process according to claim 5, further comprising the reaction of compound (6) with phosphorous oxychloride (POCl₃), followed by treatment with methanol, to give methyl 2-chloro-6-cyclopentylisonicotinate (8).

9. The process according to claim 7, further comprising the reaction of compound (8) with NaOMe/MeOH, followed by hydrolysis of the ester, to give 2-cyclopentyl-6-methoxy-isonicotinic acid (I):

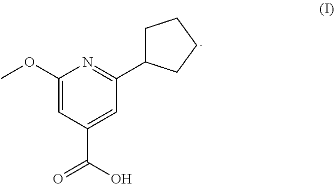 [SMILES: COc1cc(C(=O)O)cc(C2CCCC2)n1]

10. The compound ethyl 2-cyclopentyl-5-cyano-6-hydroxyisonicotinate or a salt thereof.

11. The compound 2-cyclopentyl-6-hydroxyisonicotinic acid or a salt thereof.

12. The compound methyl 2-cyclopentyl-6-hydroxyisonicotinate or a salt thereof.

13. The compound methyl 2-chloro-6-cyclopentylisonicotinate or a salt thereof.

14. A process for the preparation of the pyridine-4-yl derivatives of formula (PD), 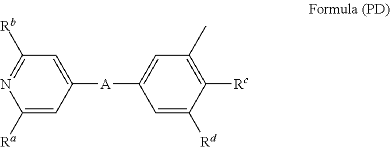
wherein
A represents 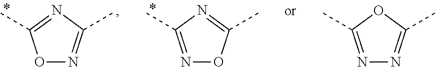
(the asterisks indicate the bond that is linked to the pyridine group of Formula (PD));
R^{a} represents cyclopentyl;
R^{b} represents methoxy;
R^{c} represents 2,3-dihydroxypropoxy, —OCH₂—CH(OH)—CH₂—NHCO—CH₂OH, —OCH₂—CH(OH)—CH₂N(CH₃)—CO—CH₂OH, —NHSO₂CH₃, or —NHSO₂CH₂CH₃; and
R^{d} represents ethyl or chloro,
comprising the process according to claim 1.

15. The process according to claim 14 for preparing a compound selected from the group consisting of:
(S)-3-{4-[5-(2-cyclopentyl-6-methoxy-pyridin-4-yl)-[1,2,4]oxadiazol-3-yl]-2-ethyl-6-methyl-phenoxy}-propane-1,2-diol;
(R)-3-{4-[5-(2-cyclopentyl-6-methoxy-pyridin-4-yl)-[1,2,4]oxadiazol-3-yl]-2-ethyl-6-methyl-phenoxy}-propane-1,2-diol;
ethanesulfonic acid {2-chloro-4-[5-(2-cyclopentyl-6-methoxy-pyridin-4-yl)-[1,2,4]-oxadiazol-3-yl]-6-methyl-phenyl}-amide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-acetamide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-N-methyl-acetamide;
N— {2-chloro-4-[5-(2-cyclopentyl-6-methoxy-pyridin-4-yl)-[1,2,4]oxadiazol-3-yl]-6-methyl-phenyl}-methane sulfonamide;
(S)-3-{2-chloro-4-[5-(2-cyclopentyl-6-methoxy-pyridin-4-yl)-[1,2,4]oxadiazol-3-yl]-6-methyl-phenoxy}-propane-1,2-diol;
N—((S)-3-{2-chloro-4-[5-(2-cyclopentyl-6-methoxy-pyridin-4-yl)-[1,2,4]oxadiazol-3-yl]-6-methyl-phenoxy}-2-hydroxy-propyl)-2-hydroxy-acetamide;
N-{4-[5-(2-cyclopentyl-6-methoxy-pyridin-4-yl)-[1,2,4]oxadiazol-3-yl]-2-ethyl-6-methyl-phenyl}-methane sulfonamide;
(S)-3-{4-[3-(2-cyclopentyl-6-methoxy-pyridin-4-yl)-[1,2,4]oxadiazol-5-yl]-2-ethyl-6-methyl-phenoxy}-propane-1,2-diol;
(R)-3-{4-[3-(2-cyclopentyl-6-methoxy-pyridin-4-yl)-[1,2,4]oxadiazol-5-yl]-2-ethyl-6-methyl-phenoxy}-propane-1,2-diol;
N—((S)-3-{4-[3-(2-cyclopentyl-6-methoxy-pyridin-4-yl)-[1,2,4]oxadiazol-5-yl]-2-ethyl-6-methyl-phenoxy}-2-hydroxy-propyl)-2-hydroxy-acetamide;
N—((R)-3-{4-[3-(2-cyclopentyl-6-methoxy-pyridin-4-yl)-[1,2,4]oxadiazol-5-yl]-2-ethyl-6-methyl-phenoxy}-2-hydroxy-propyl)-2-hydroxy-acetamide;
(S)-3-{4-[5-(2-cyclopentyl-6-methoxy-pyridin-4-yl)-[1,3,4]oxadiazol-2-yl]-2-ethyl-6-methyl-phenoxy}-propane-1,2-diol;
(R)-3-{4-[5-(2-cyclopentyl-6-methoxy-pyridin-4-yl)-[1,3,4]oxadiazol-2-yl]-2-ethyl-6-methyl-phenoxy}-propane-1,2-diol;
N—((S)-3-{4-[5-(2-cyclopentyl-6-methoxy-pyridin-4-yl)-[1,3,4]oxadiazol-2-yl]-2-ethyl-6-methyl-phenoxy}-2-hydroxy-propyl)-2-hydroxy-acetamide; and
N—((R)-3-{4-[5-(2-cyclopentyl-6-methoxy-pyridin-4-yl)-[1,3,4]oxadiazol-2-yl]-2-ethyl-6-methyl-phenoxy}-2-hydroxy-propyl)-2-hydroxy-acetamide; or salts of these compounds.

16. The process according to claim 14 for preparing a compound selected from the group consisting of:
(S)-3-{4-[5-(2-cyclopentyl-6-methoxy-pyridin-4-yl)-[1,2,4]oxadiazol-3-yl]-2-ethyl-6-methyl-phenoxy}-propane-1,2-diol;
(R)-3-{4-[5-(2-cyclopentyl-6-methoxy-pyridin-4-yl)-[1,2,4]oxadiazol-3-yl]-2-ethyl-6-methyl-phenoxy}-propane-1,2-diol;
ethanesulfonic acid {2-chloro-4-[5-(2-cyclopentyl-6-methoxy-pyridin-4-yl)-[1,2,4]-oxadiazol-3-yl]-6-methyl-phenyl}-amide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-acetamide;
N—((S)-3-{4-[5-(2-cyclopentyl-6-methoxy-pyridin-4-yl)-[1,2,4]oxadiazol-3-yl]-2-ethyl-6-methyl-phenoxy}-2-hydroxy-propyl)-2-hydroxy-N-methyl-acetamide;
N-{2-chloro-4-[5-(2-cyclopentyl-6-methoxy-pyridin-4-yl)-[1,2,4]oxadiazol-3-yl]-6-methyl-phenyl}-methane sulfonamide;
(S)-3-{2-chloro-4-[5-(2-cyclopentyl-6-methoxy-pyridin-4-yl)-[1,2,4]oxadiazol-3-yl]-6-methyl-phenoxy}-propane-1,2-diol;
N—((S)-3-{2-chloro-4-[5-(2-cyclopentyl-6-methoxy-pyridin-4-yl)-[1,2,4]oxadiazol-3-yl]-6-methyl-phenoxy}-2-hydroxy-propyl)-2-hydroxy-acetamide; and
N-{4-[5-(2-cyclopentyl-6-methoxy-pyridin-4-yl)-[1,2,4]oxadiazol-3-yl]-2-ethyl-6-methyl-phenyl}-methane sulfonamide;
or salts of these compounds.
